(19) United States
(12) Patent Application Publication
(10) Pub. No.: US 2005/0010303 A1
(43) Pub. Date: Jan. 13, 2005

(54) Acetabular cup for hip prosthesis ball-socket or the like

(76) Inventor: Alexis Nogier, Fontenay Sous Bois (FR)

Correspondence Address: YOUNG & THOMPSON, 745 SOUTH 23RD STREET, 2ND FLOOR, ARLINGTON, VA, 22202, US

(21) Appl. No.: 10/497,910

(22) PCT Filed: Nov. 28, 2002
(86) PCT No.: PCT/FR02/04076

(30) Foreign Application Priority Data
Dec. 6, 2001 (FR) 01/15759

(51) Int. Cl. A61F 2/34

(52) U.S. Cl. 623/22.26; 623/22.24; 623/22.28

(57) ABSTRACT

The present invention relates to acetabular cup assemblies for ball-and-socket joint protheses. The acetabular cup assembly is essentially characterized by the fact that it comprises a cup 20 of outside general shape that is substantially hemispherical, the cup including an open hollow housing 21, and an insert 23 in which a hemispherical cavity 24 is made suitable for receiving a spherical head to co-operate in rotation therewith, the insert being of outside shape that is substantially complementary to the housing 21 so as to be capable of being engaged therein, the cup 20 comprising a central base 30 constituting the polar cap of the cup 20, a determined number of cup portions 31-33, and means 34 for associating the cup portions 31-33 at least with the central base 30 to form the cup 20 with its housing 21. The invention is applicable to making a prosthesis, in particular a hip prosthesis or the like.

### FIELD OF THE INVENTION

The present invention relates to acetabular cup assemblies constituting the female parts of ball-and-socket joint protheses that find a particularly advantageous but non-exclusive application in making hip prostheses, for example.

### PRIOR ART

An acetabular cup assembly already exists constituting the female portion of a ball-and-socket joint prosthesis for the hip or the like, that comprises a cup of a general outside shape that is substantially hemispherical including an open hollow housing, and an insert in which a hemispherical cavity is made in which a spherical head belonging to the male portion of said ball-and-socket joint prosthesis engages to co-operate in rotation, the insert being of outside shape that is substantially complementary to the housing so as to be capable of being engaged therein.

In order to implant such an acetabular cup assembly in the cotyloid cavity of the ileum of a patient's hip, it is necessary to begin by making a large incision in the flesh surrounding the hip, and then to dislocate strongly the joint between the femur and the ileum.

The large incision also makes it possible to work on the cotyloid cavity so as to give it a hemispherical shape, e.g. by means of an auxiliary tool in the form of a spherical rasp or the like.

It is clear that the need to make a large incision in the flesh of a patient and the need to dislocate the hip strongly constitute drawbacks. The patient will be in pain after the operation, and will take longer to recover when the incision is large and the hip joint has been subjected to strong dislocation.

Present-day surgical techniques, such as those that are computer assisted, make it possible to avoid making large incisions and dislocating the hip joint in order to work on the cotyloid cavity and even to work on the medullary canal of the femur.

However, because of the size of the implant, essentially the size of the acetabular cup assembly, it continues to be necessary to make a large incision in the patient's flesh and to dislocate the joint strongly in order to be able to introduce the acetabular cup assembly.

### OBJECT OF THE INVENTION

The present invention seeks to provide an acetabular cup assembly for a ball-and socket joint prosthesis for the hip or the like which mitigates the drawback of the above-mentioned prior art acetabular cup assembly.

### SUMMARY OF THE INVENTION

More precisely, the present invention provides an acetabular cup assembly constituting the female portion of a ball-and socket joint prosthesis for a hip or the like, the cup assembly comprising:
a cup of outside general shape that is substantially hemispherical, said cup including an open hollow housing; and
an insert in which a hemispherical cavity is made in which a spherical head belonging to the male portion of the ball-and socket joint prosthesis engages to co-operate in rotation, said insert having an outside shape that is substantially complementary to the housing so as to enable it to be engaged therein,
   the acetabular cup assembly being characterized by the fact that said cup is constituted by a determined number of cup portions that are hinged to one another.

The present invention also provides an acetabular cup assembly constituting the female portion of a ball-and socket joint prosthesis for a hip or the like, the cup assembly comprising:
a cup of outside general shape that is substantially hemispherical, said cup including an open hollow housing; and
an insert in which a hemispherical cavity is made in which a spherical head belonging to the male portion of the ball-and socket joint prosthesis engages to co-operate in rotation, said insert having an outside shape that is substantially complementary to the housing so as to enable it to be engaged therein,
   characterized by the fact that said cup assembly comprises:
a central base constituting the polar cap of said cup;
a determined number of cup portions; and
first means for associating said cup portions at least with said central base to form said cup together with its housing.

### BRIEF DESCRIPTION OF THE FIGURES

Other characteristics and advantages of the invention appear from the following description given with reference to the accompanying drawings by way of non-limiting illustration, in which:

FIG. 1 is a highly diagrammatic view of a hip prosthesis implanted in a patient;

FIG. 2 is a section view showing the basic scheme for a first embodiment of the acetabular cup assembly of the invention;

FIG. 3 is a section view showing the basic scheme for a second embodiment of the acetabular cup assembly of the invention;

FIG. 4 is a fragmentary view in section showing the basic scheme for a third embodiment of the acetabular cup assembly of the invention; and

FIGS. 5 and 6 are diagrams showing two embodiments of an improvement to the embodiments of acetabular cup assemblies as shown in FIGS. 2 to 4, respectively when seen from above and in cross-section.

It should be understood that in the figures the same references are used to designate elements that are the same regardless of the figure in which they appear and the way in which the elements are shown. Similarly, if elements are not specifically referenced in one of the figures, their references can easily be found by referring to another figure.

The Applicant seeks also to make it clear that although the figures show a plurality of embodiments of the subject matter of the invention, there can also exist other embodiments that satisfy the definition of the invention.

The Applicant also specifies that when the subject matter of the invention is stated in the definition of the invention to comprise “at least one” element having a given function, the embodiment described may have a plurality of such elements.

It is also specified that where the embodiments of the subject matter of the invention as shown comprise a plurality of elements that are identical in function, and where the description does not specify that the subject matter of the invention must necessary have some particular number of such elements, then the subject matter of the invention can be defined as comprising “at least one” such element.

### DESCRIPTION OF PREFERRED EMBODIMENTS OF THE INVENTION

In order to restore a joint that is defective for medical or accidental reasons, use is often made of a joint prosthesis.

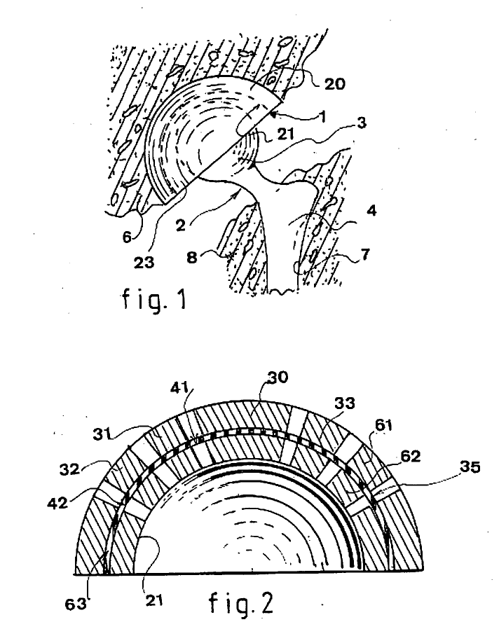

FIG. 1 is a diagram of a prosthesis 2 which is more particularly intended for restoring the joint between the femur and the corresponding cotyloid cavity of the ileum.

Such a prosthesis comprises an acetabular cup assembly which is implanted in the cotyloid cavity of the ileum 6. The acetabular cup assembly is generally constituted both by a cup 20 of substantially hemispherical outside shape, e.g. made of metal or the like, for co-operating with the ileum, said cup including an open hollow housing 21, and by an insert 23 generally made of polyethylene, ceramic, etc., said insert having an outside shape substantially complementary to the housing 21 so as to be capable of being engaged therein, preferably by force.

The insert also has an optionally lined spherical cavity 24 constituting the female portion of a ball-and-socket joint, in which a complementary spherical head 3 constituting the male portion of the ball-and-socket joint is engaged to co-operate in rotation, said head being generally secured to a prosthesis shaft 4 for implanting the medullary canal 7 of the femur 8.

FIGS. 2 to 4 show different embodiments of an acetabular cup assembly of the invention comprising a cup and an insert, but the insert is not shown in FIG. 2.

According to a characteristic of the invention, the cup 20 is constituted by a determined number of cup portions 31, 32, 33, . . . that are hinged relative to one another.

More particularly, with reference to FIGS. 2 to 4, the cup 20 advantageously comprises a central base 30 constituting the polar cap of the cup, a determined number of cup portions 31, 32, 33, . . . , and first means 34 for associating these cup portions 31-33 with at least the central base 30 so as to form the cup 20 together with its housing 21.

The central base 30 may optionally be constituted as a single piece, of relatively small size but nevertheless of sufficiently large size to provide a seat for the cup 20 in the bottom of the bony cavity in which the acetabular cup assembly is to be implanted, e.g. the cotyloid cavity of the ileum.

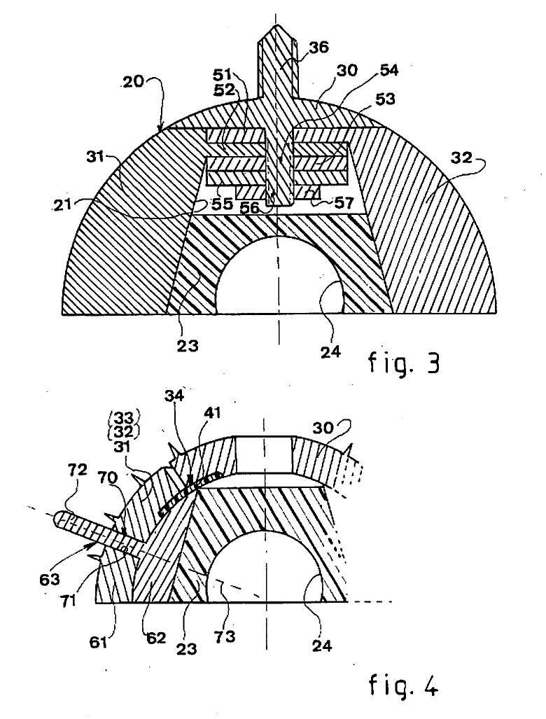

Advantageously, this central base 30 includes means for anchoring in the bone, for example a bone-thread screw 36 or the like (FIG. 3), or other mechanical means such as roughnesses, spikes, or the like, possibly coupled to chemical means such as layers of bone-bonding material which are well known in themselves.

In an advantageous embodiment (FIG. 2), the cup 20 further comprises second means 35 for associating the cup portions 31-33 with one another.

The first means 34 for associating the cup portions 31-33 with at least the central base 30 are advantageously constituted (FIGS. 2 and 4) by first flexible wall portions 41 secured respectively to each cup portion 31-33 and to the central base 30, these first flexible wall portions forming flexible hinges enabling the cup portions to pivot relative to the central base 30. Such a flexible wall can be made of a material such as polyethylene, fabric, a fine plate or trellis of metal, titanium, or the like, etc.

The second means 35 for associating the cup portions 31-33 with one another, when required as in the embodiment of FIG. 2, are constituted by second flexible wall portions 42 interconnecting the cup portions, these second flexible wall portions 42 preferably being made in the same manner as the first flexible wall portions 41 as described above.

Nevertheless, in one possible embodiment, such as the embodiment shown in FIG. 2, the first flexible wall portions 41 and the second flexible wall portions 42 are constituted as a single part.

As shown in FIG. 3, in an embodiment which presents advantages in terms of industrial manufacture, in particular, the first means 34 for associating the cup portions 31-33 with at least the central base 30 so as to form the cup with its housing 21 are constituted by a plurality of female hooks 51, 52, 53, . . . equal in number in the embodiment shown to the number of cup portions 31-33, and secured to respective ones of the cup portions 31-33, and a male hook 54 secured to the central base 30 in such a manner that, when each female hook 51-53 is hooked on the male hook, the assembled cup portions 31-33 and central base 30 form the cup 20 together with its housing 21.

However, it is also possible for one or two cup portions 31-33 to be secured to the central base 30, with the one or two cup portions and the central base 30 being machined from a single part, for example. The central base 30 then includes the male hook 54, and the cup portions other than those associated with the central base 30 include respective female hooks 51-53. In order to limit the number of female hooks, it is even possible to associate at least two of these other cup portions in common with the same female hook 51-53.

In a preferred embodiment, each female hook 51-53 is constituted by a ring 55 and the male hook 54 is constituted by a stud 56 on which the rings are suitable for being engaged stacked one on another, and by means 57 for securing the rings 55 to the stud 56. If the stud is constituted by a threaded rod, the means 57 can be constituted by a nut or the like.

In the embodiment shown in FIG. 3, there are four cup portions 31-33. They are distributed at 90° intervals relative to one another, and each is associated with a respective ring 55. The means 34 thus comprise four rings stacked on the stud 56, and these rings are associated with the cup portions 31-33 in such a manner as to be at different heights relative to one another so as to enable them to be engaged on the stud 56 while holding the cup portions in position relative to one another so that their outside faces form a continuous hemispherical surface and their inside faces form the housing 21 that is to receive the insert 23, as shown in FIG. 3.

In an embodiment which can be preferable in certain applications, it is also possible for each cup portion 31-33 to be constituted by a stack of two wall portions 61, 62, an outer wall portion 61 for forming the outer wall of the cup 20 and an inner wall portion 62 for forming the inner wall of the cup and thus the wall of the housing 21 (see FIGS. 2 and 4), together with means 63 for associating the outer and inner wall portions 61 and 62.

In a possible embodiment, these means 63 for associating the inner and outer wall portions 61 and 62 are constituted by male-female engagement means 70 (FIG. 4).

Advantageously, these male-female engagement means 70 are constituted by a through hole 71 made through the outer wall portion 61 and an oblong peg 72 secured to the inner wall portion 62, the peg being shaped to be engaged in the through hole and its length being greater than the depth of the through hole 71, e.g. so as to enable it to be implanted in the ileum 6 if the prosthesis is a hip prosthesis.

Advantageously, the axis 73 of the peg 72 is situated substantially on a radius of a hemispherical cup 20.

In an advantageous embodiment, at least one of the flexible wall portions 41, 42 is sandwiched between at least two wall portions 61, 62 and is secured thereto so as to constitute a cup portion 31-33 (see FIGS. 2 and 4).

Under such circumstances, the means 63 for associating the outer and inner wall portions 61 and 62 are constituted by said flexible wall portion itself, which is sandwiched between the outer and inner wall portions and which is secured thereto, e.g. by welding, adhesive, or the like.

As shown in FIG. 2, the flexible wall portions 41, 42 are made as a single piece and all of the cup portions formed with the outer and inner wall portions 61 and 62 together with the central base 30 are associated with this single piece by sandwiching it.

This structure gives the cup a degree of flexibility in deformation before it is implanted. The cup becomes rigid once it is implanted in the cotyloid cavity of the ileum and the insert 23 has been engaged in the housing 21, e.g. by force.

In order to implant an acetabular cup assembly of the invention as described above, the size of the incision made in the flesh of the patient is small. The cotyloid cavity and the medullary canal of the femur are preferably prepared for implantation using computer-controlled tools, implementing techniques that are nowadays known in themselves. This work can be performed not by dislocating the hip but merely by separating the hip with a small offset, e.g. by applying a tension force between the leg and the pelvis of the patient.

Once this work has been done, the cup 20 is inserted into the cotyloid cavity in its folded shape for the embodiments of FIGS. 2 and 4, and it is then unfolded to take up its final shape.

For the embodiment of FIG. 3, the cup is inserted into the cotyloid cavity piece by piece and is assembled in situ.

In the embodiment of FIG. 3, the cup is generally introduced by putting the central base 30 in place initially, e.g. by screwing the screw 36 into the bottom of the cotyloid cavity in the ileum, and then by associating the cup portions 31-33 therewith one by one by threading the rings 55 on the stud 56, and finally by screwing the nut 57 so as to complete the cup 20 in its final shape.

The insert 23 is then passed through the incision and inserted into the housing which generally does not pose any difficulty since the insert is small in size compared with the size of the cup once it has taken its final shape. The insert is then engaged in the cup, if necessary by snap-fastening, thus enabling the cup to be well defined in its final deployed shape and enabling it to be stiffened, with the cup also being held on the outside against the wall of the cotyloid cavity in the ileum.

Finally, the shank 4 of the prosthesis supporting the male spherical portion 3 of the ball-and-socket joint is introduced into the medullary canal 7 of the femur 8.

The spherical head is put into co-operation with the cavity 24 of the insert 23 by releasing the tension applied to the leg relative to the pelvis.

However, in order to avoid any tendency of the acetabular cup assembly of the invention to open like the corolla of a flower under the action of the stresses that are applied thereto when it is implanted, the acetabular cup assembly advantageously includes hoop means 80 which serve to hold the cup portions 31, 32, and 33 and prevent them from moving apart from one another like the pedals of a flower.

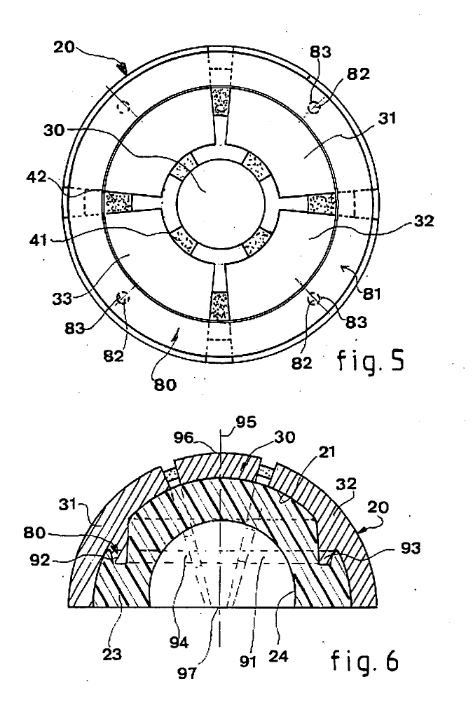

FIG. 5 shows a first embodiment of the acetabular cup assembly of the invention in which the hoop means 80 are constituted by outer banding 81 positioned on the outside surface of the deployed cup portions 31-33. This outer banding 81 is constituted by a sheath or the like forming a segment of a sphere as shown in FIG. 5, and it is made out of a biocompatible material such as stainless steel which may advantageously include, on its inside surface, and by way of example, studs 82 suitable for engaging in complementary holes 83 formed in the outside wall of the cup portions so as to ensure that the sheath does not rise up along the cup 20.

The outer banding 81 may also be constituted by an annular strand or ring or the like of biocompatible material suitable for being inserted in a circular groove made in the periphery of the cup 20.

FIG. 6 shows another embodiment of the acetabular cup assembly in which the hoop means 80 are constituted by internal banding 91. This internal banding 91 is constituted by ring portions 92, 93 secured to the inside wall of each cup portion 31-33 which form, inside the cup 20 when the cup portions 31-33 are deployed, a projecting portion 94 of substantially circularly cylindrical shape that is substantially centered on the axis 95 that passes through the pole 96 and the centers 97 of the cup 20 and of the spherical cavity 24. This circularly cylindrical projecting portion 94 is suitable for co-operating with a complementary groove formed in the outside wall of the insert 23 when it is placed in the cup, and in prior art acetabular cup assemblies.

1. An acetabular cup assembly (1) constituting the female portion of a ball-and socket joint prosthesis (2) for a hip or the like, the cup comprising:
a cup (20) of outside general shape that is substantially hemispherical, said cup including an open hollow housing (21); and
an insert (23) in which a hemispherical cavity (24) is made in which a spherical head (3) belonging to the male portion of the ball-and socket joint prosthesis (2) engages to co-operate in rotation, said insert having an outside shape that is substantially complementary to the housing (21) so as to enable it to be engaged therein,
the acetabular cup assembly being characterized by the fact that said cup (20) is constituted by a determined number of cup portions (31, 32, 33, . . . ) that are hinged to one another.

2. An acetabular cup assembly according to claim 1, characterized by the fact that said cup (20) further comprises:
a central base (30) constituting the polar cap of said cup (20); and
first means (34) for associating said cup portions (31-33) at least with said central base (30) so as to form said cup (20) with its housing (21).

3. An acetabular cup assembly according to claim 1, characterized by the fact that said cup (20) further comprises second means (35) for associating said cup portions (31-33) with one another.

4. An acetabular cup assembly according to claim 2, characterized by the fact that the first means (34) for associating said cup portions (31-33) at least with said central base (30) are constituted by first flexible wall portions (41) secured respectively to each cup portion (31-33) and to said central base (30).

5. An acetabular cup assembly according to claim 4, characterized by the fact that the second means (35) for associating said cup portions (31-33) with one another are constituted by second flexible wall portions (42) interconnecting the cup portions.

6. An acetabular cup assembly according to claim 5, characterized by the fact the first flexible wall portions (41) and the second flexible wall portions (42) are constituted by a single part.

7. An acetabular cup assembly according to claim 2, characterized by the fact that the first means (34) for associating said cup portions (31-33) at least with said central base (30) to form said cup with its housing (21) are constituted by:
a plurality of female hooks (51, 52, 53, . . . ) secured to respective ones of said cup portions (31-33); and
a male hook (54) secured to said central base (30) in such a manner that when each female hook (51-53) is hooked on said male hook the assembly comprising said cup portions (31-33) and the central base (30) forms the cup (20) together with its housing (21).

8. An acetabular cup assembly according to claim 7, characterized by the fact that each female hook (51-53) is constituted by a ring (55) and that the male hook (54) is constituted by a stud (56) on which said rings are suitable for being engaged stacked one on another, and by means (57) for securing said rings (55) with said stud (56).

9. An acetabular cup assembly according to claim 2, characterized by the fact that each cup portion (31-33) is constituted by a stack of two wall portions (61, 62), an outer wall portion (61) to form the outside wall of the cup (20) and an inner wall portion (62) to form the wall of the housing (21), and by means (63) for associating the outer and inner wall portions (61, 62) with each other.

10. An acetabular cup assembly according to claim 9, characterized by the fact that the means (63) for associating the outer and inner wall portions (61 and 62) are constituted by male-female engagement means (70).

11. An acetabular cup assembly according to claim 10, characterized by the fact that the male-female engagement means (70) are constituted by a through hole (71) made in the outer wall portion (61) and by an oblong peg (72) secured to the inner wall portion (62), said peg being shaped to engage in said through hole and its length being greater than the depth of said through hole (71).

12. An acetabular cup assembly according to claim 4, characterized by the fact that at least one of said flexible wall portions (41, 42) is sandwiched between at least two wall portions (61, 62) to constitute a cup portion (31-33).

13. An acetabular cup assembly according to claim 1, characterized by the fact that it further comprises hoop means (80) for holding the cup portions (31, 32, 33) so as to prevent them tending to spread apart from one another.

14. An acetabular cup assembly according to claim 13, characterized by the fact that the hoop means (80) are constituted by outer banding (81) positioned on the outside surface of the deployed cup portions (31-33).

15. An acetabular cup assembly according to claim 14, characterized by the fact that said outer banding (81) is constituted by at least one of the following elements: a sheath in the form of a segment of a sphere, an annular strand, a ring.

16. An acetabular cup assembly according to claim 15, characterized by the fact that said sheath includes, on its inside surface, studs (82) suitable for being positioned in complementary holes (83) formed in the outside wall of the cup portions.

17. An acetabular cup assembly according to claim 13, characterized by the fact that the hoop means (80) are constituted by inner banding (91) constituted by ring portions (92, 93) secured to the inside wall of each cup portion (31-33), forming a projecting portion (94) inside the cup (20) when the cup portions (31-33) are deployed, the projecting portion being substantially circularly cylindrical in shape and substantially centered on the axis (95) passing through the pole (96) and the centers (97) of the cup (20) and the spherical cavity (24), said circularly cylindrical projecting portion (94) being suitable, when the insert (23) is placed in the cup (20) for co-operating with a complementary groove made in the outside wall of the insert (23).

18. An acetabular cup assembly according to claim 2, characterized by the fact that said cup (20) further comprises second means (35) for associating said cup portions (31-33) with one another.

19. An acetabular cup assembly according to claim 3, characterized by the fact that the first means (34) for associating said cup portions (31-33) at least with said central base (30) are constituted by first flexible wall portions (41) secured respectively to each cup portion (31-33) and to said central base (30).

20. An acetabular cup assembly according to claim 3, characterized by the fact that the second means (35) for associating said cup portions (31-33) with one another are constituted by second flexible wall portions (42) interconnecting the cup portions.
